(19) United States
(12) Patent Application Publication
(10) Pub. No.: US 2016/0297699 A1
(43) Pub. Date: Oct. 13, 2016

(54) PSYCHROPHILIC ANAEROBIC DIGESTION OF AMMONIA-RICH WASTE

(71) Applicant: HER MAJESTY THE QUEEN IN RIGHT OF CANADA, as represented by THE MINISTER OF AGRICULTURE AND, Sherbrooke (CA)

(72) Inventor: Daniel I. MASSÉ, Sherbrooke (CA)

(73) Assignee: HER MAJESTY THE QUEEN IN RIGHT OF CANADA, AS REPRESENTED BY THE MINISTER OF AGRICULTURE AND, Sherbrooke, QC (CA)

(21) Appl. No.: 15/099,694

(22) PCT Filed: Oct. 14, 2014
(86) PCT No.: PCT/CA2014/050989

§ 371 Date: Apr. 15, 2016

Related U.S. Application Data
(60) Provisional application No. 61/892,523, filed on Oct. 18, 2013.

(51) Int. Cl. C02F 3/28; C05F 3/00; C12P 5/02; C05F 1/00; C05F 11/00; C02F 3/34; C05F 9/00

(52) U.S. Cl. CPC C02F 3/282; C02F 3/348; C05F 3/00; C05F 9/00; C05F 1/005; C05F 11/00; C12P 5/023; C02F 2103/26

(57) ABSTRACT

The present description relates to a process for the psychrophilic anaerobic digestion of ammonia-rich waste, such as farm manure or municipal waste, comprising the steps of contacting the ammonia-rich waste to an inoculum comprising anaerobic bacteria in a digester and reacting the ammonia-rich waste with the inoculum at a temperature below 25° C. to allow digestion of the ammonia-rich waste.

### TECHNICAL FIELD

The present description relates to a psychrophilic anaerobic digestion process of ammonia-rich waste.

### BACKGROUND ART

Hog production is a vital element of Canada's agricultural economy. In 2006, Canada's 11,497 pork producers raised 30.8 million pigs, in which 75% of this production happened in three provinces: Ontario (26.5%), Quebec (24.9%) and Manitoba (23.6%). In 2006, the agricultural sector in Quebec generated 7.5% of total greenhouse gas emissions (GHG), that is, 6.36 Mt of carbon dioxide equivalents, while emissions generated by swine production, due, among other things, to the spreading of pig manure as fertilizer, contributed to about 15% of total farm emissions, which represents less than 1% of total GHG emissions in Quebec. Despite the fact that the swine sector is not an important source of GHG emissions, an association is sometimes made between these emissions and odours, and ammonia, which is why the swine industry considers it necessary to promote good farming practices as a means of reducing these emissions.

Replacing fossil fuels with renewable energy is an effective manure management options to reduce the total GHG emissions for the agricultural sector (Masse et al., 2010, Bioresource Technology, 102: 641-646; Rajagopal et al., 2011, Bioresource Technology, 102: 2185-2192). Biomethanization of pig slurry consists of the microbial digestion in an oxygen-free environment of the organic matter contained in slurry, manure or other organic excretion. This reaction produces a biogas, composed mainly of methane (60%), carbon dioxide (40%) and a negligible amount of other gases. Once produced, this biogas can be burned directly in a boiler system where the hot water is used for heating buildings or, in some cases, directly in a small gas-powered electric generator. The biogas capture and methane combustion would make it possible to decrease GHG and generate carbon credits by: reducing fugitive methane emission from manure storages, recovering methane produced inside bioreactors, generating heat and other forms of energy on the farm with the biogas, which accordingly reduces the need for fossil fuels; better management of the nitrogen inside the liquid fraction (greater fertilizing efficiency) resulting from the digestion treatment, thus decreasing nitrous oxide emissions from agricultural soils.

Despite these benefits, however, digestion of concentrated swine manure (8 to 10% TS) or of poultry manure as a sole substrate has previously been shown to be unsuccessful, mainly due to its high content of ammonia (Rajagopal et al., 2012, Bioresource Technology, 14: 632-641).

One consuming way of reducing issues with treating swine manure, flushing systems have been used to remove pig manure from the swine building. This result in low TS content and swine manure becomes then not problematic for AD processes. Generally, swine manure is not problematic unless it is concentrated (10% TS) which result in high ammonia content (7 to 9 g/L). Poultry manure is problematic for AD due to its high nitrogen content (15 g to 35 g/L)

Ammonia is regularly reported as the primary cause of digester failure because of its direct inhibition of microbial activity (Hansen et al., 1998, Water Research, 33: 1805-1810; Chen et al., 2008, Bioresource Technology, 99: 4044-4064; Hejnfelt and Angelidaki, 2009, Biomass and Bioenergy, 33: 1046-1054). Ammonia is vital for bacterial growth but also hinders the anaerobic digestion (AD) process if present in high concentration. Total ammonia concentration (TAN) greater than 4 g N/L was shown to be inhibitory during digestion of livestock manure (Angelidaki and Ahring, 1993, Water Research, 28: 727-731; Sung and Liu, 2003, Chemosphere, 53: 43-52; Chen et al., 2008, Bioresource Technology, 99: 4044-4064). TAN comprises of free (un-ionised) ammonia (NH₃) [FAN] and ionized ammonium nitrogen (NH₄⁺), in which FAN has been suggested as the cause of inhibition in high ammonia loaded process since it is freely membrane-permeable (Angelidaki and Ahring, 1993, Water Research, 28: 727-731; Nielsen and Angelidaki, 2008, Bioresource Technology 99: 7995-8001). FAN concentration primarily depends on few important parameters viz. TAN, temperature, pH and ionic strength of the digesting material. Studies have suggested that increase in temperature or pH will lead to an increase in the fraction of FAN (Angelidaki and Ahring, 1993, Water Research, 28: 727-731; Sung and Liu, 2003, Chemosphere, 53: 43-52; Procházka et al., 2012, Aplied Microbiology and Biotechnology, 93: 439-447).

A study on piggery manure at 37° C. indicated that a FAN levels of about 150 mg N/L cause growth inhibition (Braun et al., 1981, Biotechnology Letters, 3: 159-164). Nakakubo et al., (2008, Environmental Engineering Science, 25: 1487-1496) observed that a 50% decrease of methane yield at a FAN levels of 1.45 g NH₃-N/L, while co-digesting pig slurry with solid fractions separated from manure. However, this study concluded that the TAN concentration seemed to inhibit the anaerobic digestion process more than the FAN levels. It has been reported that a FAN concentration of 0.69 g NH₃-N/L caused 50% inhibition of methanogenesis under thermophilic conditions (Gellert and Winter, 1997, Applied Microbiology and Biotechnology, 48: 405-410). In a similar study, Nielsen and Angelidaki (2008, Bioresource Technology, 99: 7995-8001) described that FAN concentration of 1.2 g N/L inhibited the anaerobic digestion of cattle manure at pH 7.6 at 55° C.

Several studies have concentrated on the prevention of various process imbalances, predominantly via development of different process control strategies, automation and augmentation of process monitoring. Few other studies have attempted to come up with practical solutions to avoid inhibition and harvest stable biogas production such as: (i) dilution of reactor content (Kayhanian, 1999, Environmental Technology, 20: 355-365; Nielsen and Angelidaki, 2008, Bioresource Technology, 99: 7995-8001); (ii) addition of materials- such as bentonite, glauconite and phosphorite with ion exchange capacity (Krylova et al., 1997, Journal of Chemical Technology and Biotechnology, 70: 355-365; Hansen et al., 1999, Water Research, 33: 1805-1810); (iii) Struvite precipitation (Maqueda et al., 2003, Water research, 28: 411-416) and use of carbon fiber textiles (Sasaki et al., 2011, Applied Microbiology and Biotechnology, 90: 1555-1561); (iv) adjustment of the feedstock C/N ratio and pH (Kayhanian, 1999, Environmental Technology, 20: 355-365; Strik et al., 2006, Process Biochemistry, 41: 1235-1238); and (v) lowering temperature from thermophilic (55° C.) to more moderate conditions [40-50° C.] (Angelidaki and Ahring, 1994, Water Research, 28: 727-731). However, some of these techniques either had a significant negative effect on methane production or economically not feasible; and none of these control techniques have been successfully implemented on the farm scale.

Total ammonia concentration (TAN) greater than 4 gN/L was shown to be inhibitory during digestion of livestock manure (Angelidaki & Ahring, 1993, Applied Microbiology and Biotechnology, 38(4): 560-564; Chen et al., 2008, Bioresource Technology, 99: 4044-4064). Accordingly, the long-term successful operation of an AD process at higher ammonia concentrations (i.e. >5 g N/L) has not yet been reported.

There is thus still a need to be provided with a way of digesting and processing ammonia-rich waste.

### SUMMARY

In accordance with the present description there is now provided a process for the psychrophilic anaerobic digestion of ammonia-rich waste comprising the steps of contacting the ammonia-rich waste to an inoculum comprising anaerobic bacteria adapted to high ammonia concentration in a digester and reacting the ammonia-rich waste with the inoculum at a temperature below 25° C. to allow digestion of the ammonia-rich waste.

In an embodiment, the ammonia-rich waste is reacted with the inoculum at a temperature of between 10 to 25° C.

In another embodiment, the ammonia-rich waste is reacted with the inoculum at a temperature of 20° C.

In an embodiment, the digestion is conducted in total ammonia N (NH₃+NH₄⁺) levels of at least 7.5 g N/L.

In an embodiment, the digestion is conducted in ammonia N (NH₃ +NH₄⁺) levels of at least 12 g N/L.

In a further embodiment, the ammonia-rich waste comprises a total nitrogen content (NH₃ +NH₄⁺+organic nitrogen) exceeding 10 000±900 mg N/I.

In a supplemental embodiment, the ammonia-rich waste comprises a total nitrogen content (NH₃+NH₄⁺+organic nitrogen) exceeding 12 900±900 mg N/I.

In an embodiment, the ammonia-rich waste is liquid waste, semi-liquid waste or solid waste.

In another embodiment, the ammonia-rich waste comprises between 8-45% of total solids content.

In a further embodiment, the ammonia-rich waste is animal manure, animal slurry, agri-food waste, slaughterhouse wastes, municipal waste, or energy crops.

In an embodiment, the animal manure is farm waste.

In an embodiment, the farm waste is dairy manure, beef manure, poultry manure, spoiled hay, silage, swine manure or cash crops.

In another embodiment, the farm waste any livestock manures (sheeps, goats, etc).

In another embodiment, the farm waste is chicken manure or pig manure.

In a further embodiment, the slaughterhouse wastes are feather, beef hoofs, blood, contaminated meat or a mixture thereof.

In a further embodiment, the process described herein comprises the further step of feeding the digester with inoculum from same or a separate silo.

In an embodiment, the inoculum is feed in batch, semi-continuously or continuously into the digester.

In another embodiment, the process described herein comprises the step of feeding the ammonia-rich waste into the digester comprising the inoculum.

In an embodiment, the ammonia-rich waste is feed in batch, semi-continuously or continuously into the digester.

In a further embodiment, the process described herein comprises the step of premixing the inoculum with the ammonia-rich waste and feeding said premixed inoculum and ammonia-rich waste into the digester.

In an embodiment, the premixed inoculum and ammonia-rich waste are feed in batch, semi-continuously or continuously into the digester.

In an embodiment, the digester is a batch reactor, a sequential batch reactor or a plug flow digester.

In another embodiment, methane is recuperated during digestion of the ammonia-rich waste.

In another embodiment, a fertilizer is recuperated from the digester after digestion of the ammonia-rich waste.

### BRIEF DESCRIPTION OF THE DRAWINGS

Reference will now be made to the accompanying drawings, showing by way of illustration, a preferred embodiment thereof, and in which:

FIG. 1 illustrates cumulative methane production following digestion as described herein of ammonia-rich swine mannure.

FIG. 2 illustrates acetic (C₂), propionic (C₃), butyric (C₄), iso-butyric (iC₄), valeric (C₅), iso-valeric (iC₅), caproic (C₆) and pH in the mixed liquor during one cycle of (A) digestion as described herein in a reactor with TAN levels of 8.2±0.3 g/L, (B) control reactor with TAN levels of 5.5±0.7 g/L.

FIG. 3 illustrates the cumulative methane production during digestion of ammonia-rich manure.

FIG. 4 illustrates the VFA composition and pH in the mixed liquor for PADSBRs with TAN levels of 11-12 g/L, (A) Ammonia concentration increased from 10 g/L to about 12 g/L; (B) New pig manure; (C) Change of pig manure; (D) Cycle length increased from 4 to 6 weeks.

FIG. 5 illustrates the VFA composition and pH in the mixed liquor for PADSBRs with TAN levels of 10 g/L; (A) Ammonia concentration increased from 8 g/L to about 10 g/L; (B) New pig manure; (C) Change of pig manure; (D) Cycle length increased from 4 to 6 weeks.

FIG. 6 illustrates the VFA composition and pH in the mixed liquor for PADSBRs with PM+CM congestion (8 gN/L); (A) Pig manure feeding along with NH₄Cl replaced by PM+CM co-digestion; (B) New pig manure; (C) Change of pig manure; (D) Cycle length increased from 4 to 6 weeks.

### DETAILED DESCRIPTION

It is provided a psychrophilic anaerobic digestion process of ammonia-rich waste, such as animal manure, that can be integrated for example in a farm waste management to potentially increase farmers income while reducing the environmental footprint of the operation.

It is disclosed a psychrophilic anaerobic digestion in sequencing batch reactor (PADSBR) to treat swine manure spiked with ammonium chloride. Ammonia inhibition was induced by pulsing with NH₄Cl to laboratory-scale PADSBRs to simulate the sharp increase in TAN levels up to 8.2±0.3 g N/L that may occurs in actual centralized biogas plants when proteinaceous co-substrates are fed to the reactors.

Essentially, it is described a psychrophilic anaerobic digestion in sequencing batch reactor (PADSBR) of ammonia-rich waste such as animal manure, animal slurry, agri-food waste, slaughterhouse wastes, municipal waste, or energy crops. The animal manure can be farm waste such as for example dairy manure, beef manure, poultry manure, spoiled hay, silage, swine manure or cash crops. Essentially, as encompassed herein, the farm waste treated can be of any livestock manures (sheeps, goats, etc).

Ammonia nitrogen plays a critical role in the performance and stability of anaerobic digestion (AD) of ammonia-rich wastes like animal manure. Nevertheless, inhibition due to high ammonia remains an acute limitation in AD process. A successful long-term operation of AD process at high ammonia levels (>5 g N/L) is limited.

The present disclosure described a psychrophilic anaerobic digestion in a sequencing batch reactor (PADSBR) to treat swine manure with excess total ammonia levels of 8.2±0.3 g N/L. The results show that total chemical oxygen demand (CODt), soluble chemical oxygen demand (CODs), volatile solids (VS) removals of 86±3, 82±2 and 73±3 were attained at an organic loading rate (OLR) of 3 gCOD/L.d. Higher ammonia had no effect on methane yields (0.23±0.04 L CH₄/gTCOD_{fed}) and are comparable to that of control reactors, which fed with pig manure only (5.5 gNH₃-N/L). Longer solids and hydraulic retention times in PADSBRs enhanced the biomass acclimation even at high NH₃-N levels. Thus volatile fatty acid (VFA), an indicator for process stability, did not accumulate in the digester. The likely inhibition by free ammonia was insignificant since the calculated values (184 mg/L) were far below the inhibitory limits reported in the art.

The psychrophilic anaerobic digestion (PAD) in sequential batch reactor (SBR), developed at Agriculture and Agri-Food, Dairy and swine Research and Development Centre (DSRDC) in Sherbrooke, Quebec-Canada for the stabilization of agricultural wastes, successfully reduces odors, decreases the organic pollution load by more than 70% (Masse et al., 1996, Canadian Journal of Civil Engineering, 23: 1285-1294), produces high quality biogas, significantly diminishes pathogens survival (Masse et al., 2011, Borescource Technology, 102: 641-646), and improves the agronomic value of digestate (Masse et al., 2007, Bioresource Technology, 98: 2819-2823).

The process offers the competitive advantages of great stability, robustness, maximum performance, and minimum supervision. Moreover, less energy is required to maintain the temperature in the digester as compared to mesophilic and thermophilic anaerobic digestion. The process uses bacteria adapted to thrive at low temperature (Dhaked et al., 2010, Waste Management, 30: 2490-2496) and digest organic substrates with total solids (TS) contents lower than 12%, such as swine manure. Low temperature wet anaerobic digestion provides a unique, very stable and cost effective process for digesting liquid swine manure.

Canadian patent no. 2,138,091 describes psychrophilic anaerobic digestion of animal manure slurry in intermittently fed sequencing batch reactors. A similar psychrophilic anaerobic digestion process as described in Canadian patent no. 2,138,091 has also been demonstrated to be able to remove hydrogen sulphide content from the biogas produced during digestion (see WO 2012/061933) and to degrade prions contained in the starting material to be digested (see WO 2011/152885).

A PAD process is described herein for the first time for agricultural wastes with ammonia-rich content.

This is the first report on successful psychrophilic dry anaerobic digestion of ammonia-rich content. It is demonstrated the feasibility of digesting ammonia-rich waste in a sequencing batch reactor.

It is thus disclosed a process for the psychrophilic anaerobic digestion of ammonia-rich waste comprising the steps of contacting the ammonia-rich waste to an inoculum comprising anaerobic bacteria in a digester and reacting the ammonia-rich waste with the inoculum at a temperature below 25° C., representing psychrophilic conditions.

Psychrophilic conditions are known to reflect bacteria activity at a temperature of about 10° C. to about 25° C.

Ammonia is the end-product of anaerobic digestion of proteins, urea and nucleic acids. Unlike the importance of ammonia for bacterial growth at lower concentration, high concentration of ammonia may cause a severe disturbance in the anaerobic process performance i.e. cause an important decrease of microbial activities. Inhibition of the AD process is usually indicated by the decrease in the steady state methane production rates and increase in the intermediate digestion products like volatile fatty acid (VFA) concentrations. Toxicity is manifested by a total cessation of methanogenic activity.

Ammonia-rich waste is intended to mean waste with a total nitrogen content exceeding 4000 mg N/L. Preferably, as demonstrated herein, the process described herein can digest ammonia-rich content of 8000 mg N/L. This level of nitrogen concentration results in bioreactor failure with some AD technologies.

Total ammonia nitrogen (TAN) is intended to mean the non-organic forms of nitrogen (ammoniac (NH3) and ammonium (NH₄⁺)). Total nitrogen include the total ammonia nitrogen as well as the organic nitrogen (proteins, amino acids) usually called TKN. TKN is always larger than TAN.

The digestion can be conducted in total ammonia N (NH₃+NH₄⁺) levels of at least 7.5 g N/L., even at least 12 g N/L. In a further embodiment, the ammonia-rich waste comprises a total nitrogen content (NH₃+NH₄⁺+organic nitrogen) exceeding 10 000±900 mg N/L, even exceeding 12 900±900 mg N/L.

Encompassed herein are the digestion of ammonia-rich liquid waste, semi-liquid waste or high solids content waste, not only farm manure and slurry such as dairy manure (cow manure), beef manure, poultry manure or swine manure, slaughterhouse wastes and agri-food waste, for example, but also municipal waste with ammonia rich content. High solids content waste are generally intended as waste having between 8-45% TS.

The process described herein also allows recuperating inoculum at the end of the digestion process in order to be stocked in a silo or reuse in the digester in a semi-continuous or continuous process.

Accordingly, the inoculum from the same digester can be used as described herein. At the end of the treatment cycle, the treated liquid effluent is removed from the bioreactor and a new batch of high nitrogen liquid substrate is fed to the bioreactor. In the case of dry AD of high nitrogen substrate the solid inoculum would come from the same bioreactor and premixed with a new batch of solids substrates prior feeding the bioreactor. The solid inoculum could be diluted and stored in a separate silo and reused to inoculate a new batch of solid substrate in the bioreactor. It is recirculated from the separate silo into the digester.

Then inoculum can be feed continuously from a separate silo into the digester. When the bioreactor is operated with liquid waste, the inoculum is already in the bioreactor and the high nitrogen substrate is fed to the bioreactor (in batch, semi-continuously or continuously). In the case the waste is solid, the inoculum is premixed with the high nitrogen content solid substrate prior feeding the bioreactor. Alternatively, the described process also comprises an inoculum reservoir where the diluted inoculum can be batch, intermittently or continuously fed to the dry solids bioreactors.

Fertilizer can also be recuperated at the end of the process. The fertilizer can then be used to supplement farm fields for example.

The reactor/digester system used herein can be a batch reactor, a sequential batch reactor or a plug flow type where the waste moves horizontally from one end to the other, the waste entering the digester which in turn, displaces digester volume, thereby causing an equal amount of material to exit from the digester.

Four laboratory-scale PADSBRs spiked with concentrated ammonia were monitored for more than a year to assess their reliability and stability in terms of organic matter removal, VFA elimination and biogas production. The average OLR applied to the bioreactors was in the range of 3 g COD/L.d, with a TCOD concentration in the feed around 146.71 g O₂/L. The pH of raw manure was about 6.91 (near neutrality), although high VFA concentrations of 22.1 g/L were detected, mostly because of the high amount of alkalinity (-22 g CaCO₃/L) in the manure.

Four PADSBRs (R1-R4) were pulsed with NH₄Cl together with the addition of swine manure to study the effects of high ammonia concentration in the digestate. Whereas, reactors R5 and R6 were kept as control digesters without the addition of excess ammonia nitrogen. The total ammonia concentrations in the reactors R1-R4 were increased to a value of 8.2±0.3 g NH₃-N/L compared to 5.5±0.7 g NH₃-N/L for the control reactors (R5-R6).

The PADSBRs (R1-R4) and control reactors (R5-R6) were operated in parallel under similar operating conditions as presented in Table 1.

TABLE 1
Operating conditions of the PADSBRs

| No of | | Operation | Sludge | | Quantity | Cycle | Fill and |
|---|---|---|---|---|---|---|---|
| replicate | | temperature | volume | OLR (g | of manure | length | react |
| ASBRs | Substrate | (° C.) | (L) | COD/L.d) | fed (L) | (week) | phase |
| 4 | Pig manure + | 24.5 ± 0.5 | 20 | 3.0 ± 0.35 | 3.9 ± 1.3* | 4 | 14 d |
| | addition of NH₄Cl | | | | | | (each) |
| 2 | Pig manure only | | | | | | |
| (control) | | | | | | | |

*fluctuation depends on the manure from different periods

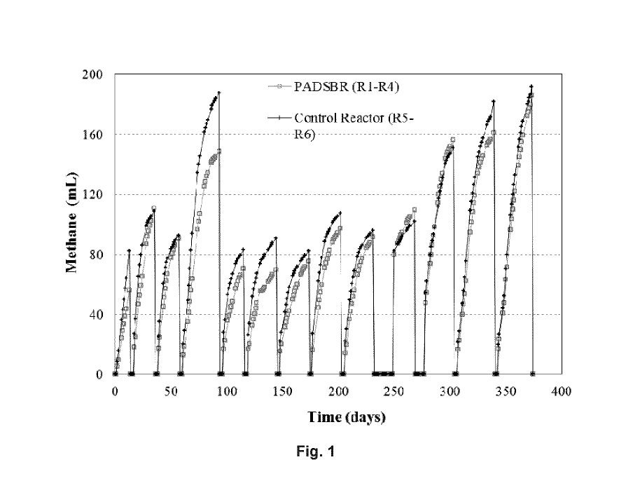

The summary of the results obtained for the removal of organics such as TCOD, SCOD, TS and VS in the treated liquor along with methane production is given in Table 2 and an illustration of the profile for the cumulative methane production is illustrated in FIG. 1.

TABLE 2
Removal of organic fractions and methane production

| | | Period of | Reduction efficiency, % | | | | Methane | CH₄ |
|---|---|---|---|---|---|---|---|---|
| | OLR, | operation, | removal | | | | yield, L CH₄/g | content of |
| Reactors | g COD/L.d | days | TCOD | SCOD | TS | VS | TCOD_{fed}^{a} | biogas (%) |
| R1-R4 | 3.0 ± 0.35 | 375 | 86 ± 3 | 82 ± 2 | 67 ± 4 | 73 ± 3 | 0.23 ± 0.04 | 68.3 ± 2.4 |
| | | | | | | | (0.48 ± 0.09)^{b} | |
| R5-R6 | | | 88 ± 1 | 84 ± 2 | 77 ± 4 | 84 ± 3 | 0.24 ± 0.05 | 70.2 ± 2.9 |
| (control) | | | | | | | (0.49 ± 0.10)^{b} | |

^{a}Values corresponding to the last 5 cycles
^{b}Values in parenthesis ( ) indicate methane yield based on VS loading (L CH₄/g VS_{fed})

Similar profiles were attained for the PADSBRs pulsed with NH₄Cl to that of control reactors with regard to COD removal efficiencies, cumulative methane production and methane yield. However, solids removals were relatively higher in the control reactors (Table 2). The probable reason could be that in PADSBRs, organic matter is reduced by biological conversion into methane and by physical removal during the settling period (Masse et al., 2008, Bioresource Technology. 99: 7307-7311). Since there is no significant differences observed in the methane production for all the reactors, differences in solids reductions may be due to the variances in physical removal. The composition of biogas with methane content of 68-70% showed that the biogas obtained during digestion of ammonia-rich manure was of good quality. Even if the pH was not controlled in the bioreactors there was no formation of foam and scum observed during this study period. The mode of operation (process, temperature) and the appropriate choice of acclimatized inoculum at the start-up of experiment allowed a high-stabilization of pig manure digestion even at high ammonia concentrations (8.2±0.3 g NH₃-N/L). Relatively higher values for the cumulative methane production after day 275 (FIG. 1) than the initial periods showed that the active biomass accumulated in the settled sludge enriched the performance of PADSBRs with time. Effective sedimentation occurred in the PADSBRs, which reduced substantially the biomass washout in the effluent. Thus, for the OLR studied (i.e. 3 COD/L.d), the addition of excess ammonia nitrogen to the pig manure did not affect the stability and performance of the PADSBRs.

AD instability can happen due to the accumulation of VFA concentrations with a concurrent decrease in methane gas production. Hence, the fate of different components of VFA was followed primarily to investigate the possibility of methanogens inhibition.

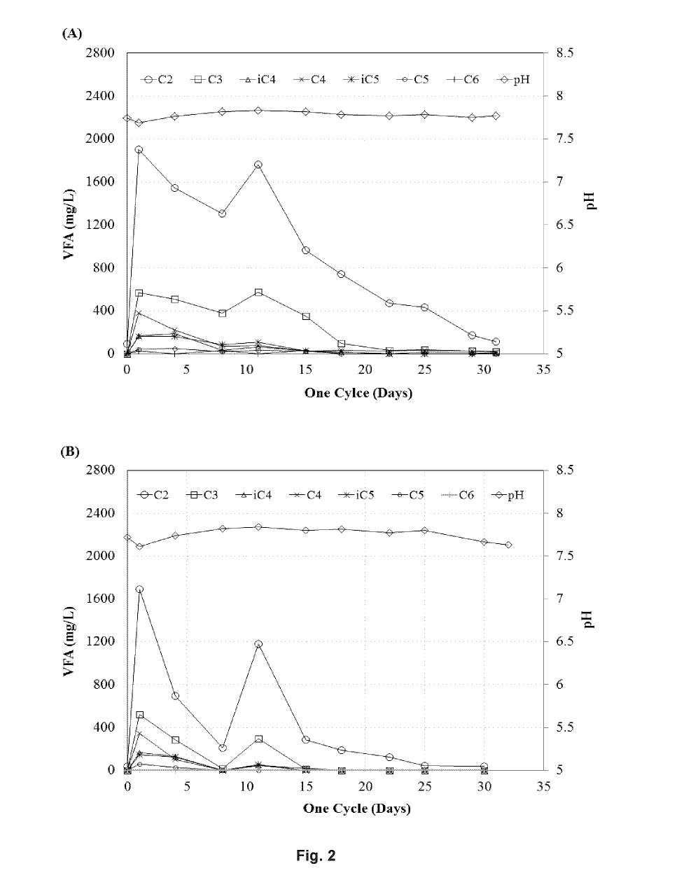

FIG. 2A and B illustrates the pH and the typical profiles of short chain fatty acids (SCFAs) such as acetic (C₂), propionic (C₃), butyric (C₄), iso-butyric (iC₄), valeric (C₅), iso-valeric (i0₅) and caproic (C₆) during one cycle of operation (4 weeks) for the PADSBRs (in the mixed liquor) with and without addition of excess ammonia. Similar VFA dynamics were observed in all the digesters but with different values. Acetic acid was the predominant VFA component produced during the digestion of pig manure, which comprised more than 73 and 85% of the total VFAs for the PADSBRs (with excess ammonia addition) and the control reactors, respectively. Whereas, propionic acid contained about 15 and 7% of the total VFAs produced, respectively and the higher molecular weight VFAs (C₄-C₆) were produced in negligible amounts (FIG. 2). As expected, higher VFA concentrations were observed just after the time of feeding (i.e. on day 0 and 7) due to the hydrolysis of complex molecules and acidogenesis, and also partly due to the high VFA concentrations in the swine manure fed to the bioreactors, as indicated in the FIG. 2. Total VFAs produced (maximum of 3235 mg/L) in the beginning of a four week cycle were eliminated towards the end (VFA<100 mg/L), showed that VFAs did not accumulate in the PADSBR by increasing ammonia N concentrations. Acclimatized methanogens allowed to consume most of the SCFAs produced within 15-18 days. Swine manure is a highly buffered waste and hence alkalinities in all the digesters were found to be optimal with an average value of 25,058±2634 and 26,322±2701 mg CaCO₃/L for the PADSBRs (R1-R4) and control digesters (R5-R6), respectively. A small deviation of less than one pH unit during cycles was observed as shown in FIG. 2, which could be explained by the high buffering capacity of swine manure.

Lauterbock et al. (2012, Water research, 46: 4861-4869) observed the accumulation of VFA, especially propionic acid, as well as the decline of biogas production while digesting slaughterhouse waste, especially when the TAN concentration exceeds 6 gNH₄-N/L at 38° C. and pH of 8.1. For a cattle manure digestion in a CSTR, Angelidaki and Ahring (1994, Water Research, 28: 727-731) witnessed that high ammonia concentration (FAN >700 mg/L) inhibited the methane production at thermophilic temperatures (55 and 64° C.) and resulted in a rapid increase in VFA concentrations (5000 mg/L) at pH 7.9. Similar results were observed using thermophilic UASB reactors by Borja et al. (1996, Process Biochemistry, 31: 477-483), in which the VFA concentrations increased from 1000 to 3000 mg/L as acetic acid with increase in ammonia concentrations up to 7 g N/L. When swine manure was anaerobically digested at temperatures from 37 to 60° C., the amount of VFA increased with increasing temperature from 4800 to 15,800 mg-acetate/L (Hansen et al., 1998, Water research, 32: 5-12). In contrast, in the PADSBR the VFAs, an indicator for the process stability, was much lower and a higher gas yield associated with enhanced degradation was observed in the present study. The psychrophilic SBR approach offers an attractive know-how to improve the process efficiency in anaerobic digestion of ammonia rich wastes.

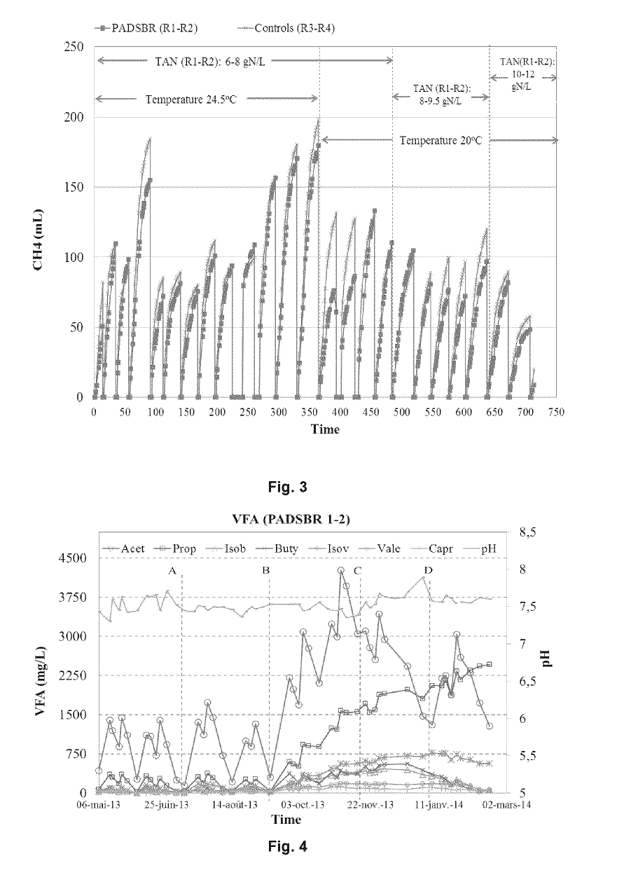

An illustration of the profile for the cumulative methane production is presented in FIG. 3. Similar profiles were attained for the PADSBRs pulsed with NH₄Cl to that of control reactors with regard to cumulative methane production and methane yield. However, solids removals were relatively higher in the control reactors. The probable reason could be that in PADSBRs, organic matter is reduced by biological conversion into methane and by physical removal during the settling period. Since there is no significant differences observed in the methane production for all the reactors, differences in solids reductions may be due to the variances in physical removal and likely effect of NH₄Cl salt used as a source of ammonia nitrogen in PADSBRs. The composition of biogas with methane content of 68-70% showed that the biogas obtained was of good quality. Average methane yield of 0.21 and 0.24 L CH₄/g TCOD_{fed} was obtained for PABSBRs (R1-R2 @ 11-12 gN/L) and controls (R3-R4), respectively. Even if the pH was not controlled in the bioreactors there was no formation of foam and scum observed during this study period. The mode of operation (process, temperature) and the appropriate choice of acclimatized inoculum at the start-up of experiment allowed a high-stabilization of pig manure digestion even at high total ammonia concentrations (11-12 g N/L).

The fluctuations in COD value and hence the cumulative methane production were due to change in manure characteristics. However, relatively higher values for the cumulative methane production from day 275-364 (FIG. 1) than the initial periods showed that the active biomass accumulated in the settled sludge enriched the performance of PADSBRs with time. Nevertheless, when we changed the temperature from 24.5 to 20° C., i.e. after day 365, the PADSBRs (R1-R2) with higher ammonia levels recorded comparatively lower methane production to that of control digesters for the two consecutive cycles of operation, i.e. from day 365-422 (FIG. 3). The fact is that a drop in digester operating temperature could probably decelerate the microbial activity especially at higher TAN levels and disturb the treatment efficiency. This is associated with the biomass loss in terms of mixed liquor VSS concentrations that could disrupt the PADSBR process by affecting biomass retention. Thanks to the long-term adaptation of microbes, this helped to recover the process stability after a drop in operating temperature. Afterwards, effective sedimentation occurred in the PADSBRs, which reduced substantially the biomass washout in the effluent. Thus, for the studied OLR (i.e. 3 COD/L.d), the addition of excess ammonia nitrogen to the pig manure did not affect the stability and performance of the PADSBRs.

An increment of total ammonia levels [NH₃+NH₄⁺] was observed in all the reactors such that average initial NH₃—N concentration augmented from 7.9 and 5.0 g/L to 8.3 and 6.3 g/L for the PADSBRs (R1-R4) and controls (R5-R6), respectively. This increase was probably due to (i) conversion of some organic nitrogen (mainly protein and urea) to ammonia during AD (Gonzalez-Fernandez and Garcia-Encina, 2009, Biomass and Bioenergy, 33: 1065-1069); (ii) accumulation of NH₃-N as more manure was fed to the bioreactors (Masse et al., 2003, Bioresource Technology, 89: 57-62). Similar profiles were observed for the TKN concentrations with average values significantly increased from 8.9 and 5.7 g/L to 9.7 and 7.8 g/L for the PADSBRs and controls, respectively.

It is likely that inhibition by ammonia in the AD process should also be related to the FAN concentrations rather than TAN or ammonium ions, as it is considered to be the foremost reason for inhibition of methane-producing consortia. Average FAN concentrations in the PADSBRs and control digesters were observed in the range of 184 and 147 mg/L, respectively. FAN concentration was calculated by using ionization equation (Eqs. 1 and 2, see Example 1) and taking pKa of 9.26 for 24.5° C. (digester temperature). The control digesters showed relatively lower FAN levels than PADSBRs, however, in our study ammonia levels were significantly lower than the threshold concentrations reported in previous inhibition works (Hansen et al., 1998, Water Research, 32: 5-12; Nakakubo et al., 2008, Environmental Engineering Science, 25: 1487-1496). As shown herein, free ammonia levels contained about 2.27 and 2.61% of total NH₃-N, i.e. sum of NH₃-N and NH₄⁺-N concentrations. Furthermore, some studies have shown that high levels of free ammonia has been proven to cause accumulation of VFA components, indicate an imbalanced microbiological activity and propionate degradation when the total ammonia concentration is around 4.0-5.7 g/L (Koster et al., 1988, Biological Wastes, 25: 51-59; Karakashev et al., 2005, Applied and Environmental Microbiology, 71: 331-338; Resch et al., 2011, Bioresource Technology, 102: 2503-2510), but again as disclosed herein the excess ammonia N did not affect the PADSBR process.

Typically, swine manures contain approximately 4-5g N/L on average. AD inhibition by ammonia reported to occur at the TAN concentrations of 1.5 -2.5 g/L (Van Velsen, 1979, Water Research, 13: 995-999; Hansen et al., 1998, Water Research, 32: 5-12). The fraction of free (undissolved) ammonia increases with temperature and pH (Sung and Liu, 2003, Chemosphere, 53: 43-52), which is commonly believed to be the actual toxic agent than ammonium ions as it is capable to penetrate through the cell membrane. In this study, without pH adjustments of the digested pig slurry (pH 7.8), degradation of propionate, butyrate and valerate (FIG. 2) as well as methane production (FIG. 1) were still feasible regardless of its high TAN concentration of 8.2 gN/L. The lower final acetate and propionate concentrations indicated that the acetoclastic methanogens and the syntrophic propionate-degrading acetogenic bacteria-hydrogenotrophic microorganisms were not inhibited at FAN levels of 184 mg/L and pH of 7.8. Similar observations were reported by Ho and Ho (2012) by reducing the initial manure pH from 8.3 to 6.5 but with a final FAN concentration of about 425 mg/L.

Low temperature digestion process shown to have a lower FAN levels than mesophilic and thermophilic conditions. The methanogens are capable of adaption to high ammonia concentrations when increasing the concentration slowly over a longer period. However, an inhibitive threshold of 1.1 g/L of FAN levels was reported by Hansen et al. (1998, Water Research, 32: 5-12) for mesophilic and thermophilic conditions with biomass adapted to high ammonia concentrations over a long period. Under thermophilic conditions, Ho and Ho (2012, Water Research, 46: 4339-4350) observed the inhibition levels of free ammonia from 916 to 643 mg N/L with an accumulation of acetate and propionate at pH from 8.3 to 7, respectively.

However, the successful process reported herein shows that methanogens in PADSBR are capable of adaption to higher concentration of ammonia (8.2 g N/L). The longer solids and hydraulic retention times in PADSBRs enhanced the biomass acclimation at these reported TAN levels. Free ammonia and VFA levels were low, illustrating that the performance of PADSBR was stable and efficient throughout the study period.

It is demonstrated herein that PADSBR technology can be employed to limit ammonia inhibition even at higher concentrations. Increasing ammonia N levels up to 8.2 g N/L did not affect the anaerobic digestion of pig manure. The mode of operation (process, temperature) along with the choice of acclimatized inoculum ensured a high-stabilization of the digestion process without inhibition and thus VFA components did not accumulate in the digester. Free ammonia levels (184 mg/L) were significantly lower than the inhibitory limits reported in the art.

It is thus disclosed herein a successful operation of PADSBR up to 10 gN/L which shows that methanogens in the digester are capable of adaption to higher concentration of ammonia at 20° C. and a pH of around 7.5. The longer solids and hydraulic retention times in PADSBRs enhanced the biomass acclimation at these reported TAN levels. In addition, PADSBR has proved to be a less energy intensive technology and is certainly be an attractive option for the farms, as the requirements for the reactor mixing and heating is considerably fewer.

Present study demonstrated the robustness of PADSBR technology that can be employed to limit ammonia inhibition even at higher concentrations (10 gN/L). The mode of operation (SBR process, temperature) along with the acclimation of biomass ensured a high-stabilisation of the digestion process without inhibition at this ammonia level. In addition, PADSBR showed a good stability with chicken manure as a co-substrate. This showed that the microflora developed in the PADSBR with time proved to be very efficient, which can sustain TKN and ammonia concentrations up to 11.5 and 10 g/L, respectively. For higher TAN levels of 12 gN/L (R1-R2), no inhibition was reported. This result shows that acclimatized biomass are expected to sustain higher TKN and TAN levels.

The present disclosure will be more readily understood by referring to the following examples which are given to illustrate embodiments rather than to limit its scope.

### EXAMPLE I

Experimental Setup and Design

The fresh raw manure slurry was collected from a manure transfer tank on a commercial swine operation located in Sherbrooke, Quebec province of Canada. The manure was screened to remove particles larger than 3.5 mm to avoid the operational problem especially plugging of the influent line with the small scale digesters. The manure was then mixed to prepare homogenize feed samples and stored in a cold room at 4° C. to prevent biological activity. NH₄Cl was chosen as the source of ammonia primarily to minimize the pH effect of ammonia addition. The inoculum was sourced from the on-going pilot scale reactor located in our laboratory, which was already acclimatized to the treatment of swine manure slurry. Average manure and inoculum characteristics during the experimental period are given in Table 3.

TABLE 3
Properties of swine manure and inoculum

| Parameter | Swine Manure | Inoculum |
|---|---|---|
| Total COD (g/L) | 146.71 ± 24.5 | 18.08 ± 2.9 |
| Soluble COD (g/L) | 42.22 ± 6.0 | 5.87 ± 0.1 |
| Total solids (g/L) | 10.5 ± 2.0 | 1.9 ± 0.2 |
| Volatile solids (g/L) | 8.7 ± 2.2 | 1.0 ± 0.1 |
| Fixed Solids (g/L) | 1.9 ± 0.3 | 0.9 ± 0.1 |
| Total VFA (g/L) | 22.1 ± 4.3 | 0.14 ± 0.01 |
| TKN (g/L) | 8.4 ± 0.6 | 5.1 ± 0.4 |
| NH₃—N (g/L) | 6.3 ± 0.4 | 4.1 ± 0.2 |
| pH | 6.91 ± 0.2 | 7.80 ± 0.1 |
| Alkalinity (g CaCO₃/L) | 22.0 ± 2.4 | 18.1 ± 0.7 |
| Phosphorous (g/L) | 1.9 ± 0.1 | 0.58 ± 0.4 |

The anaerobic fermentation of swine manure was performed using psychrophilic anaerobic digestion in sequencing batch reactors (PADSBRs). Four identical (replicates) PADSBRs were used to study the effect of excess ammonia concentrations on the AD process, whereas two (replicates) PADSBRs were kept as control, which fed with pig manure only. PADSBRs were installed at a controlled-temperature room, adjusted at a temperature of 24.5±0.5° C. The sludge volume in the all the reactors were maintained at 20-L and the OLRs were based on the amount of COD_{fed} (g TCOD_{fed}) per L of sludge. All the reactors were operated for more than one year and the operating conditions are presented in Table 1.

A typical operation cycle length consists of four weeks which included the fill, react and draw phases. The feeding was carried out on day 0 and 7 of each cycle. Mixing was done by recirculating the biogas using a dual-head air pump twice a week for about 5 minutes just before taking mixed liquor samples for analysis. To simulate more suitable operational conditions on a commercial farm, no external mixing was employed. The fill and react periods duration were two weeks each, for a total treatment duration of 4 weeks. During the fill and react phases, the soluble organics and some of the suspended organic particulates are transformed into inorganic carbon by the anaerobic microorganisms. At the end of every four week cycle (i.e. end of react phase), the settling of biomass was completed and the supernatant (treated) wastewater was drawn out from the PADSBRs leaving 20-L sludge volume before feeding with fresh manure. This operating strategy was followed for the consecutive cycles of operation. OLR was maintained around 3 g COD/L.d throughout the experiment. Daily biogas production was measured using wet tip gas meters.

A mixed liquor samples of 100 mL capacity was taken biweekly from the PADSBRs after 5 minutes of mixing by recirculating the biogas. At the end of each cycle (i.e. four weeks), the settled biomass and the supernatant (treated) effluent were also collected for their physico-chemical characteristic analysis. Raw swine manure was sampled during the filling period. These samples were analysed for TCOD, SCOD, TS, VS, VFAs (acetic, propionic, butyric, etc.), pH, alkalinity, TKN and NH₃-N.

The pH value was measured immediately upon collection of samples using PH meter (model, TIM840, France). TCOD and SCOD were determined according to the method developed by Knechtel (1978). SCOD of fresh manure and effluent samples was determined by analyzing the supernatant of slurry samples after centrifugation. VFAs concentration was determined using a Perkin Elmer gas chromatograph model 8310 (Perkin Elmer, Waltham, Mass.), mounted with a DB-FFAP high resolution column. Before VFAs quantification, samples were conditioned according to the procedures described by Masse et al. (2011, Bioresource Technology, 102: 641-646). Alkalinity, TS and VS were determined using standard methods (APHA, 1992). TKN and NH₄—N were analyzed using a Kjeltec auto-analyzer model TECATOR 1030 (Tecator AB, Hoganas, Sweden) according to the macro-Kjeldahl method (APHA, 1992). Daily biogas production was measured using wet tip gas meters. Every week, biogas composition (methane, carbon dioxide, and nitrogen) was determined with a HachCarle 400 AGCgas chromatograph (Hach, Loveland, Colo.). The column and thermal conductivity detector were operated at 80° C. The nitrogen content was subtracted from the results, because N₂ gas was used as a filler gas during drawdown.

Free ammonia level was calculated according to Koster (1986). It was reported that the fraction of free ammonia relative to the TAN is dependent on pH and temperature, as reported in Eqs. (1) and (2). The percentage of free ammonia to that of total concentration was determined using Eq. (3)

$$\begin{matrix}{NH}_{2} ( Free ) - TAN ( \frac{1}{1 + 1 {e}^{- ( pKa - pH )}} ) & ( 1 ) \\ pKa = 0.08018 + ( \frac{2728.82}{T} ) & ( 2 ) \\ {NH}_{3} \% = \frac{[ {NH}_{3} ] \times 100}{[ {NH}_{3} ] + [ {NH}_{4}^{+} ]} & ( 3 )\end{matrix}$$

NH₃: Free ammonia nitrogen (FAN), mg/L;
NH₄⁺: Ammonium ion, mg/L;
TAN: Total ammonia nitrogen, mg/L;
pKa: Equilibrium ionization constant; and
T(K): Temperature (Kelvin).

### EXAMPLE II

### Psychrophilic Anaerobic Digestion in Sequencing Batch Reactor of Manure with Excess Ammonia Nitrogen

The anaerobic fermentation of swine manure was performed using tweleve identical (replicates) PADSBRs (R1-R12), in order to study the effect of excess ammonia concentrations on the AD process. In which, four (replicates) PADSBRs were used to study the co-digestion of pig manure (PM) and chicken manure (CM). PADSBRs were installed at a controlled-temperature room, adjusted at a temperature of 20±0.5° C. The sludge volume in all the reactors were maintained at 20-L (effective volume, 24 L) and the OLRs were based on the amount of COD_{fed} (gTCOD_{fed}) per L of sludge. Operating conditions are presented in Table 4.

TABLE 4
Operating conditions of the PADSBRs

| No of | | | | | | Cycle | |
|---|---|---|---|---|---|---|---|
| replicate | | Operation | Sludge | OLR, | Quantity of | length, | Fill and react |
| ASBRs | Substrate | temperature, ° C. | volume, L | g COD/L.d | manure fed, L | week | period |
| 2 | Pig manure + | 20 ± 0.5 | 20 | 2.0-3.0 | 3.9 ± 1.3* | 4 | Fill: Day 0 |
| | addition of | | | | (for one | | and 7 of each |
| | NH₄Cl | | | | cycle) | | cycle |
| | (~12 gN/L) | | | | | | React: 4 and 3 |
| 6 | Pig manure + | | | | | | weeks of each |
| | addition of | | | | | | cycle** |
| | NH₄Cl | | | | | | |
| | (~10 gN/L) | | | | | | |
| 4 | Pig and | | | | | | |
| | chicken | | | | | | |
| | manure co- | | | | | | |
| | digestion | | | | | | |
| | (7.5-8.5 gN/L) | | | | | | |

*Fluctuation depends on the manure collected at different periods
**For a 4 week cycle length, the react periods of 4 and 3 weeks corresponds to fill period t = 0 and t = 7 days, respectively

A typical operation cycle length consists of four weeks which included the fill, react, settle and draw phases. The fill step involves the addition of swine manure to the PADSBR system. The feeding was carried out on day 0 and 7 of each cycle and the feed volume was determined on the basis of desired OLR used in this study. During the react phase, the soluble organics and some of the suspended organic particulates were transformed into biogas by the anaerobic microorganisms. At the end of every four week cycle (i.e. end of react phase), the settling of biomass was completed and the supernatant (treated) wastewater was drawn out from the PADSBRs leaving 20-L sludge volume before feeding with fresh manure. The volume decanted is normally equal to the volume fed during the fill step. The high food to microorganism (F/M) ratio occurred immediately after feeding step resulted in high-rate of substrate utilization and hence, high-rate of waste conversion to biogas. Whereas, towards the end of react phase, the F/M ratio was at its lowest level with low biogas production, provided ideal conditions for biomass settling and thus enhanced longer solids (biomass) retention time.

Mixing was done by recirculating the biogas using a dual-head air pump twice a week for about 5 minutes just before taking mixed liquor samples for analysis purpose only. Otherwise, no additional external mixing was employed primarily to simulate more suitable operational conditions on a commercial farm. This operating strategy was followed for the consecutive cycles of operation. OLR was maintained around 2-3 gCOD/L.d throughout the experiment. Daily biogas production was measured using wet tip gas meters.

PADSBRs spiked with higher ammonia levels were monitored to assess their reliability and stability in terms of VFA elimination, organic matter removal and biogas production. The average OLR applied to the bioreactors was in the range of 2-3 g COD/L.d, with a TCOD concentration in the feed around 146.7 g 0₂/L. The pH of raw manure was about 6.91 (near neutrality), although high VFA concentrations of 22.1 g/L were detected, mostly because of the high amount of alkalinity (-22 g CaCO₃/L) in the manure. Average manure and inoculum characteristics during the experimental period are given in Table 5.

TABLE 5
Properties of swine manure and inoculum

| Parameter | Swine Manure | Inoculum |
|---|---|---|
| Total COD (g/L) | 146.7 ± 24.5 | 18.1 ± 2.9 |
| Soluble COD (g/L) | 42.2 ± 6.0 | 5.8 ± 0.1 |
| Total solids (g/L) | 10.5 ± 2.0 | 1.9 ± 0.2 |
| Volatile solids (g/L) | 8.7 ± 2.2 | 1.0 ± 0.1 |
| Fixed Solids (g/L) | 1.9 ± 0.3 | 0.9 ± 0.1 |
| Total VFA (g/L) | 22.1 ± 4.3 | 0.14 ± 0.01 |
| TKN (g/L) | 8.4 ± 0.6 | 5.1 ± 0.4 |
| NH₃—N (g/L) | 6.3 ± 0.4 | 4.1 ± 0.2 |
| pH | 6.91 ± 0.2 | 7.80 ± 0.1 |
| Alkalinity (g CaCO₃/L) | 22.0 ± 2.4 | 18.1 ± 0.7 |
| Phosphorous (g/L) | 1.9 ± 0.1 | 0.58 ± 0.4 |

The PADSBRs (R1-R2) were spiked with NH₄Cl together with the addition of swine manure to study the effects of high ammonia concentration up to 12 g N/L in the digestate; whereas, reactors (R3-R8), ammonia concentration was maintained in the range of 10 g N/L. PADSBRs (R9-R12), chicken manure, which is rich in ammonia, was used as a co-substrate to digest pig manure. As chicken manure contains high ammonia, no external addition of NH₄Cl was done for those PADSBRs.

All the PADSBRs were operated in parallel under similar operating conditions as presented in Table 4. AD instability can happen due to the accumulation of VFA concentrations with a concurrent decrease in methane gas production. Hence, the fate of different components of VFA was followed primarily to investigate the possibility of methanogens inhibition.

FIGS. 4-6 illustrates the pH and the typical profiles of short chain fatty acids (SCFAs) such as acetic (C₂), propionic (C₃), butyric (C₄), iso-butyric (iO₄), valeric (C₅), iso-valeric (iC₅) and caproic (C₆) for the representative PADSBRs (in the mixed liquor) i.e. with ammonia concentration of 11-12 gN/L, 10 gN/L and PM+CM codigestion, respectively. Similar VFA dynamics were observed in all the digesters but with different values. Acetic acid was the predominant VFA component produced during the digestion process. Whereas, propionic acid was found to be higher in the digesters (R1-R2) especially after September 2013 onwards. As expected, higher VFA concentrations were observed just after the time of feeding (i.e. on day 0 and 7) due to the hydrolysis of complex molecules and acidogenesis, and also partly due to the high VFA concentrations in the swine manure and/or chicken manure fed to the bioreactors, as indicated in the FIGS. 4-6.

For PADSBRs (R1-R12), total VFAs produced in the beginning of a four week cycle were almost eliminated towards the cycle end until September 2013. It is to be noted that from September 2013, a new pig manure was used with lower total COD content of about 104 g/L instead of 146±24.5 g/L in previous cycles. To compensate the organic matter difference fed to the reactors, the volume of the feed was increased accordingly. In addition to this, there were probably some unknown inhibition occurred using this new pig manure, which might have disturbed the stability and performance of the digesters from September-December 2013.

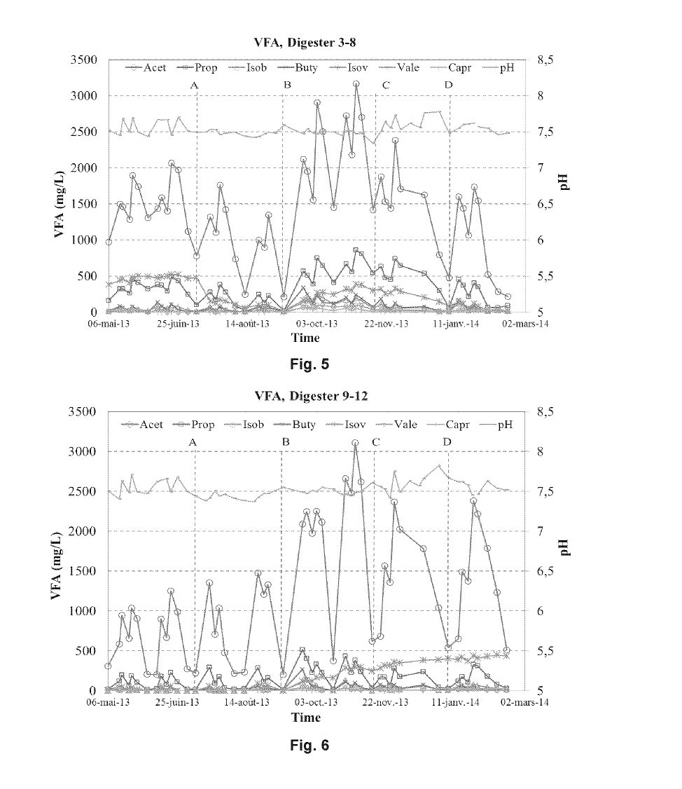

To overcome this situation, from November 2013 onwards a new pig manure from a different pig farm was used and also operation cycle for January 2014 was extended to 6 weeks instead of 4 weeks for this particular cycle of operation. The cycle was increased primarily to get rid of accumulated VFAs in the digesters. As indicated in the FIGS. 4-6, the SCFAs were started dropping in the all the digesters except propionic acid concentrations in R1-R2. This result show that, digesters R1-R2 need some more time to eliminate propionic acids compared to other digesters. Swine manure is a highly buffered waste and hence alkalinities in all the digesters were found to be optimal with an average value of 25,058±2634. A small deviation of less than one pH unit during cycles was observed as shown in FIG. 5, which could be explained by the high buffering capacity of swine manure. Co-digestion of pig and chicken manure showed a good stability in terms of VFA elimination (FIG. 6). However, the accumulation of isovaleric acid needs to be monitored with time.

The summary of the results especially NH₄-N, TKN concentrations, free ammonia, methane yield and its composition is presented in Table 6.

TABLE 6
Synopsis of results obtained from April 2013-February 2014

| | | Total ammonia | Avg. | | | | CH₄ |
|---|---|---|---|---|---|---|---|
| | | [NH₃ + NH₄⁺] | TKN | FAN | % of FAN to | CH₄ yield, L | content of |
| Reactors | Substrate | (g/L) | (g/L) | (mg/L) | TAN | CH₄/g VS_{fed} | biogas, % |
| R1-R2 | Pig | 11-12 | 12.9 ± 0.9 | 105-174 | 0.90-1.32 | 0.23 ± 0.08 | 70 ± 3 |
| | manure + NH₄Cl | | | | | | |
| | addition | | | | | | |
| R3-R8 | Pig | 9-10 | 11.5 ± 0.6 | 97-124 | 1.03-1.33 | 0.39 ± 0.10 | 71 ± 5 |
| | manure + NH₄Cl | | | | | | |
| | addition | | | | | | |
| R9-R12 | Pig + Chicken | 7.5-8.5 | 10.0 ± 0.9 | 84-109 | 0.96-1.24 | 0.25 ± 0.08 | 61 ± 4 |
| | manure | | | | | | |

The results show that the addition of excess total ammonia nitrogen (up to 12 g N/L) or total kheldahl nitrogen (TKN) 12.9±0.9 g N/L to the pig manure did not affect the stability and performance of the PADSBRs. However, comparatively lower values of methane yield for the PADSBRs, R1-R2 and R9-R12 were observed; which probably explained by the (i) higher ammonia levels using NH₄CI addition and the effect of higher TKN concentrations present in the chicken pellet used as a co-substrate, respectively and (ii) new feedstock used during September 2013, which probably caused some unknown inhibition. The composition of biogas with methane content of 61-70% showed that the biogas obtained was of good quality. Hence, the active biomasses accumulated in the settled sludge are expected to improve the performance of PADSBRs with time. Even if the pH was not controlled in the bioreactors there was no formation of foam and scum observed during this study period. Effective sedimentation occurred in the PADSBRs, which reduced substantially the biomass washout in the effluent. The mode of operation (process, temperature) and the appropriate choice of acclimatized inoculum at the start-up of experiment allowed a stabilisation of pig manure digestion even at high total ammonia concentrations (10 g N/L).

It is likely that inhibition by ammonia in the AD process should also be related to the free ammonia nitrogen (FAN) concentrations rather than TAN or ammonium ions, as it is considered to be the foremost reason for inhibition of methane-producing consortia. Average FAN concentrations in the PADSBRs were observed in the range of 84 and 174 mg/L (Table 6). FAN concentration was calculated by using ionization equation (Eqs. 1 and 2) and taking pKa of 9.40 for 20° C. (digester temperature).

While the invention has been described in connection with specific embodiments thereof, it will be understood that it is capable of further modifications and this application is intended to cover any variations, uses, or adaptations of the invention following, in general, and including such departures from the present disclosure as come within known or customary practice within the art and as may be applied to the essential features hereinbefore set forth, and as follows in the scope of the appended claims.

What is claimed is:

1. A process for the psychrophilic anaerobic digestion of ammonia-rich waste comprising the steps of:
a) contacting the ammonia-rich waste to an adapted inoculum comprising anaerobic bacteria in a digester and
b) reacting the ammonia-rich waste with the inoculum at a temperature below 25° C. to allow digestion of the ammonia-rich waste.

2. The process of claim 1, wherein the ammonia-rich waste is reacted with the inoculum at a temperature of between 10 to 25° C.

3. The process of claim 1 or 2, wherein the ammonia-rich waste is reacted with the inoculum at a temperature of 20° C.

4. The process of any one of claims 1-3, wherein the digestion is conducted in ammonia N levels of at least 5 g N/L.

5. The process of any one of claims 1-4, wherein the digestion is conducted in ammonia N levels of at least 7.5 g N/L.

6. The process of any one of claims 1-5, wherein the digestion is conducted in ammonia N levels of at least 12 g N/L.

7. The process of any one of claims 1-6, wherein the ammonia-rich waste comprises a total nitrogen content exceeding 10 000±900 mg N/I.

8. The process of any one of claims 1-7, wherein the ammonia-rich waste comprises a total nitrogen content exceeding 12 900±900 mg N/I.

9. The process of any one of claims 1-8, wherein the ammonia-rich waste is liquid waste, semi-liquid waste or solid waste.

10. The process of any one of claims 1-9, wherein the ammonia-rich waste comprises between 8-45% of total solids content.

11. The process of any one of claims 1-9, wherein the ammonia-rich waste is animal manure, animal slurry, agri-food waste, slaughterhouse wastes, municipal waste, or energy crops.

12. The process of any one of claims 1-11, wherein the animal manure is farm waste.

13. The process of claim 12, wherein the farm waste is dairy manure, beef manure, poultry manure, spoiled hay, silage, swine manure or cash crops.

14. The process of claim 12, wherein the farm waste is chicken manure or pig manure.

15. The process of claim 11, wherein the slaughterhouse wastes are feather, beef hoofs, blood, contaminated meat, rendering or a mixture thereof.

16. The process of any one of claims 1-15, comprising the further step of feeding the inoculum into the digester from a separate silo.

17. The process of claim 16, wherein the inoculum is feed in batch, semi-continuously or continuously into the digester.

18. The process of any one of claims 1-15, comprising the step of feeding the ammonia-rich waste into the digester comprising the inoculum.

19. The process of claim 18, wherein the ammonia-rich waste is feed in batch, semi-continuously or continuously into the digester.

20. The process of any one of claims 1-15, comprising the step of premixing the inoculum with the ammonia-rich waste and feeding said premixed inoculum and ammonia-rich waste into the digester.

21. The process of claim 20, wherein said premixed inoculum and ammonia-rich waste are feed in batch, semi-continuously or continuously into the digester.

22. The process of any one of claims 1-21, wherein the inoculum is recuperated at the end of the digestion.

23. The process of any one of claims 1-22, wherein the digester is a batch reactor, a sequential batch reactor or a plug flow digester.

24. The process of any one of claims 1-23, wherein methane is recuperated during digestion of the ammonia-rich waste.

25. The process of any one of claims 1-24, wherein a fertilizer is recuperated from the digester after digestion of the ammonia-rich waste.
